(19) United States
(12) Patent Application Publication
(10) Pub. No.: US 2014/0074077 A1
(43) Pub. Date: Mar. 13, 2014

(54) Method for Reducing the Absorption of Nutrients Through the Gastrointestinal Tract

(71) Applicant: Sanford Lane, Sherborn, MA (US)

(72) Inventor: Sanford Lane, Sherborn, MA (US)

(73) Assignee: GASTRO-SHAPE TECHNOLOGIES, INC, Geneva, CH (CH)

(21) Appl. No.: 14/004,051

(22) PCT Filed: Mar. 9, 2012
(86) PCT No.: PCT/US12/28451

§ 371 Date: Nov. 22, 2013

Related U.S. Application Data
(60) Provisional application No. 61/450,904, filed on Mar. 9, 2011.

(51) Int. Cl. A61B 18/22; A61B 18/14; A61F 5/00

(52) U.S. Cl. CPC A61B 18/22; A61F 5/0076; A61B 18/1482
USPC 606/15

(57) ABSTRACT

The present invention features methods for applying a force, such as laser or radiofrequency (RF) energy to the wall of the small intestine, which targets and eliminates a portion of the capillary and vascular network responsible for the transportation of nutrients to the patient. This causes the patient to absorb a reduced percentage of nutrients, which results in a reduced caloric intake. Various types and shapes of applicators are used to deliver the treatment energy, and these applicator devices are within the scope of the present invention. The applicator can be deployed through either a body cavity (e.g., the oral cavity), an open surgical procedure, or a minimally invasive incision or incisions.

### CROSS-REFERENCE TO RELATED APPLICATIONS

This application claims the benefit of the priority date of U.S. provisional application No. 61/450,904, which was filed Mar. 9, 2011. For any U.S. patent or application that claims priority to the present application, the content of this earlier filed provisional application is hereby incorporated by reference herein in its entirety.

### FIELD OF THE INVENTION

The present invention features medical devices, kits and methods for reducing the absorption of nutrients through the gastrointestinal tract by applying energy, for example a radiofrequency signal or laser, to the wall of the small intestine. The methods can be used to help maintain an individual's weight or to treat overweight or obese individuals.

### BACKGROUND

The percentage of the world's population suffering from obesity or morbid obesity is steadily increasing. It is well established that obese people are susceptible to increased risk of serious medical conditions, including heart disease, stroke, diabetes, and pulmonary disease, and even mild obesity increases these risks. There are also difficult social and societal implications, and overweight people are more prone to accidental injury. Because obesity affects a patient's life so greatly, many methods of treatment are being used and many others are being researched. For some patients, dietary modification alone can lead to successful weight loss but achieving or maintaining a consistent and healthy weight remains difficult for many people.

Many types of non-operative therapies for obesity are available, but these therapies rarely result in permanent weight loss and, in some cases, produce unwanted side effects or complications for the patient. These therapies include dietary counseling, hypnosis, behavior modification, and pharmacological intervention. In other instances, mechanical devices are inserted into the body through nonsurgical means. For example, gastric balloons can be used to fill the stomach. Such devices, however, cannot be deployed over a long period of time as they can cause irritation, including ulcerations, which necessitates their periodic removal. This causes temporary or permanent interruption of treatment. For reasons such as these, the medical community has adopted more aggressive surgical approaches, particularly for treatment of morbid obesity.

Many surgical procedures for treating morbid obesity are directed toward the prevention of normal absorption of food (malabsorption) and/or a restriction of the stomach to make the patient feel full sooner (gastric restrictions). A common technique is gastric bypass surgery. In variations of this technique, the stomach is divided into two isolated pouches, with the upper pouch having a reduced food capacity. The upper pouch is then connected to the jejunum through a small stoma. This reduces the size of the stomach, reduces the extent to which food can be processed in the stomach, and reduces absorption from the intestine (by bypassing the duodenum). Other procedures remove portions of the small intestine or shunt segments of the small intestine to reduce the amount of nutrients absorbed into the body. These procedures are considered major surgery, as they constitute extensive and permanent reconstruction of the gastrointestinal (GI) tract, and they carry with them all of the operative and postoperative complications and risks of open abdominal surgery.

One procedure, gastric banding, involves the placement of a polymer or metallic band around a portion of the GI tract. The band can be sized and positioned to constrict flow to or within the stomach or other parts of the GI tract, and the extent of the constriction can vary depending on a given patient's condition. However, in each case the band remains as an implanted device, and the surgeon may also implant a separate device that is connected to the band and capable of adjusting it. Typically, gastric banding is performed in an open surgical environment. There are minimally invasive techniques, but these can be problematic. Another disadvantage of gastric banding is that the patients are reluctant to undergo procedures in which a device is left in the body.

### SUMMARY

The present invention is based, in part, on our discovery of a minimally invasive, non-mechanical means for reducing nutrient absorption into the body through the gastrointestinal (GI) tract. Unlike existing methods such as gastric bypass and gastric banding, which result in significant physical or mechanical alterations of anatomical structures and pathways, the present methods inhibit nutrient absorption into the body, thereby reducing caloric consumption and promoting weight loss or weight maintenance, without reconstruction of the GI tract or attachment of mechanical appliances.

In one aspect, the invention features medical devices for delivering energy to the lumen of the intestine. The devices include: (a) an elongated housing having a proximal end and a distal end; (b) an energy-delivery channel; and (c) a tissue expander at the distal end of the housing. The energy-delivery channel can: extend from the proximal end of the housing toward the distal end; be configured to transport energy or guide an energy conduit; and terminate beyond the distal end of the housing in an energy-emitting element (that is, the energy-delivery channel and the energy-emitting element are operably linked).

As will be apparent to one of ordinary skill in the art, the terms “proximal” and “distal” are relative terms, signifying the location of one element relative to another. “Proximal” refers to the portion of an item (e.g., a device as a whole; a housing therefor, or a channel running through the device) that is nearest the operator (e.g., a physician). “Distal” refers to the portion of the item that is furthest from the operator. “Proximal” and “distal” may even be used to designate the relative positions of two elements within the same end of an item. For example, we describe devices that include, at their distal end, two tissue expanders. Even though both are found at the distal end of the device, the tissue expander that is nearest the operator is the proximal tissue expander and the tissue expander that is furthest from the operator is the distal tissue expander. An element that is said to be located in the distal region of an item or at a distal end, may or may not be the most distal element of the item.

The elongated housing can be rigid or semi-rigid. It can vary in length from about 1 foot to about 25 feet, and it can have a diameter suitable for insertion through a working channel of an endoscope or a trocar.

The energy conduit can include an optical fiber, wire, or transducer, and the energy delivery channel or the energy conduit can include a connector for attaching the channel or the conduit to an energy source (e.g., a laser, radiofrequency generator, ultrasound generator, or cryogenic probe). The energy-emitting element can be fixed such that energy emanates from the element in a diffuse pattern. Alternatively, the energy-emitting element can be focused and/or moveable such that energy emanates from the element toward a focused point, which point can vary depending on how the element is focused and/or how the element is moved. “Diffuse” can mean completely isotropic radiation or directional radiation that has a strong peak intensity in one direction and some relatively small portion (side lobes) emanating in various other directions (a common energy distribution in radiofrequency beams). The latter is generally considered a focused beam. Thus, the energy-emitting element can produce a focused beam with side lobes.

Any of the devices described herein can include a plurality of tissue expanders. For example, a device can include first and second tissue expanders. The first tissue expander can be located distal to the energy-emitting element, and the second tissue expander can be located proximal to the energy-emitting element. Either or both of the first and second tissue expanders can be shaped as a sphere, an ellipse, a ring, or a cone; either or both can be inflatable (e.g., an inflatable balloon).

Any of the devices described herein can include a visualization channel, which may run substantially parallel to the energy-delivery channel and include a scope, which is optionally moveable, attached to or integrated with the visualization channel at or near the distal end of the visualization channel. An optical element, such as a lens or filter, can be positioned over the aperture.

Any of the devices described herein can include a working channel, which may be used to administer electrolytes to the intestinal lumen, to insert surgical instruments that may facilitate manipulation of the tissue, or to apply a material (a fluid or gas) that can modulate (e.g., reduce) the temperature of the tissue during treatment. The working channel can run substantially parallel to the energy-delivery channel and can be configured to transport a fluid or gas of a given temperature to the tissue to which energy has been applied by the device.

Any of the devices described herein can include, preferably near the distal end of the housing and/or near the tissue targeted for treatment, a sensor for determining a physiological parameter, such as temperature.

In another aspect, the invention features kits that include a device as described herein and instructions for use. The kits can further, optionally, include materials to facilitate the assembly, disassembly, or sterilization of the devices.

In another aspect, the invention features methods of reducing the amount of nutrients that are absorbed into the vascular system of the small intestine of a subject. The methods can be carried out by providing a device as described herein; positioning the energy-emitting element of the device within the lumen of the small intestine of the subject; and applying energy from the device to the internal surface of the small intestine. The energy is of a type and delivered for a time sufficient to inhibit the absorption of nutrients from the treated portion of the small intestine. Positioning the energy delivery device can be accomplished by inserting the energy-emitting element of the device into the intestine through a laparoscope positioned in the subject's abdominal cavity or an endoscope positioned in the subject's upper GI tract. The medical device can deliver laser energy, and the method can further include application of an electrolyte solution between the energy-emitting element and the surface of the intestinal tissue being treated. A device can be connected to a power source supplying energy with a power of about 0.1 to about 50 watts/cm² (e.g., power of at least or about 25, 30, 35 or 40 watts/cm²). Where the medical device delivers laser energy, it can include or be connected to a power source supplying energy with a pulse width of about 1 ms to about 10 sec (e.g., at least or about 0.01, 0.5, 1.0, 1.5, or 2.0 sec). Where the medical device delivers laser energy, it can include or be connected to a power source supplying energy with a pulse configuration of about 1 pps to about 1,000 pps and, in some embodiments up to about 10,000 pps with a reduction in pulse width. Where the medical device delivers radiofrequency energy, it can include or be connected to a power source supplying energy with a power of about 1 to about 100 watts/cm² (e.g., about 25, 30, 35 or 40 watts/cm²). Where the medical device delivers radiofrequency energy, it can include or be connected to a power source supplying energy with a pulse width of about 1 ms to about 10 sec (e.g., at least or about 0.1, 0.5, 1.0, 1.5, or 2.0 sec). Where the energy delivery device delivers radiofrequency energy, it can include or be connected to a power source supplying energy with a pulse configuration of about 1 pps to about 1,000 pps.

The treatment can be “semi-permanent” in that the vasculature in the subject may return to the absorption capacity it had prior to treatment over the course of about six to twelve weeks. The methods may reduce the percentage of the nutrients in the small intestine that are absorbed into the vascular network of the small intestine, thereby reducing the subject's caloric intake.

The subject can be a mammal (e.g., a human).

The methods can further include the step of collecting, through a sensor placed adjacent to the treated tissue, data that provides feedback useful in determining whether the amount of energy supplied to the tissue is sufficient. The data can be obtained by visualization, impedance, ultrasound, or temperature measurement, which may be obtained by a device located outside of or separate from the medical device. The data can be obtained periodically throughout the treatment method.

While the invention is not limited to methods and devices that achieve reduced caloric absorption by any particular physiological mechanism, the expectation is that blood flow within the treated intestinal tissue (e.g., the tissue responsible for absorption of nutrients) is impaired. For example the applied energy may seal, collapse, narrow, and/or eliminate a portion of the vascular and lymphatic structures.

The energy input can vary so long as it produces the desired outcome of reduced caloric absorption. For example, the energy input can be configured as an electrosurgical signal or a laser, and the energy applied to the target tissue (including, for example, lymphatic ducts, capillaries, other blood vessels, and blood) affects the tissue and/or blood, resulting in reduced nutrient absorption. For example, the treatment can result in about a 10-20% reduction in caloric absorption immediately after treatment relative to absorption before treatment or to absorption from an untreated intestine. The temperature can be monitored (e.g., with a thermal sensor) and the effects of energy absorption can also be monitored (e.g. by a visual inspection as the procedure is being carried out). While the energy may be delivered in the context of conventional surgery, where the intestine is accessed through a conventional incision, an advantage of the present method is that the energy emitting portion of a device (e.g., a transducer at an applicator tip or fiber-optic) can be guided to the intestine through a mechanical guide placed through a smaller incision (e.g., the guide can be a conventional device used in surgery, such as a catheter, trocar, or laparoscope). In such circumstances, the incision can be minimized. Treatment through the abdominal wall may be preferred where the stomach has been surgically reconfigured. In many instances, however, the energy can be applied by way of a device inserted through the oral cavity (e.g., within a channel of an endoscope).

The type of energy applied can vary, as described further below. For example, devices useful in the present methods include those that emit electromagnetic energy. More specifically, the emitted energy can be radiofrequency energy (e.g., where the energy-emitting element of the device comprises an electrode or array of electrodes for transmission of radio frequency electrical current), microwaves (e.g., where the energy-emitting element comprises a microwave antenna), light or laser energy (e.g., where the energy-emitting portion of the device comprises an optical waveguide or optical fiber) or sound energy (e.g., ultrasound). However, in all cases, the energy, when applied to tissue in the intestinal tract, permanently or semi-permanently reduces caloric uptake; the functionality of the treated vessels, with respect to the absorption of nutrients and calories, is reduced. Laser treatment may be advantageous because laser energy at certain wavelengths will pass through the mucosal lining without damaging it and be absorbed by underlying structures, such as blood vessels and lymphatic ducts.

The amount of tissue affected and the degree to which any tissue within a treatment area is affected can vary depending on the extent of the treatment. For example, the treatment may be applied to only about 4-6 inches along the length of the intestine (e.g., within the duodenum). Where more aggressive treatment is required, the treatment can be applied to a greater length, for example, about 10-15 inches along the length of the intestine (e.g., of the duodenum). The degree to which any tissue within a treatment area is affected can also vary by varying the amount of energy applied and/or the length of the treatment. The more tissue that is treated and the more intense the treatment, the greater the reduction in the absorption of nutrients and calories. For example, the extent of the treatment will depend on the energy configuration (i.e., the power applied, the time, the phase, and the configuration of any pulsed energy (e.g., the frequency, amplitude, and the pulse). The proximal portion of the duodenal lining is initially smooth, but as the duodenum moves further from the stomach, the lining acquires folds and mall projections (villi and microvilli). The villi and microvilli increase the surface area of the duodenal lining, allowing for greater absorption of nutrients.

Although the methods of the invention can be directed to the duodenum, the remainder of the small intestine, including the jejunum and the ileum, can also be treated. These sections of the small intestine also absorb fats and other nutrients. The intestinal wall is supplied with blood vessels that carry the absorbed nutrients to the liver through the portal vein. Small amounts of enzymes that digest proteins, sugars, and fats are also released along with mucus and water, which lubricates the intestinal contents, which helps dissolve the digested fragments. All of these factors contribute to the process of digestion, which ultimately leads to the absorption of nutrients by the vascular system. While the invention is not so limited, we expect treatment of the small intestine to be effective when the duodenum is targeted because this region both absorbs fats and also sends signals to the more distal regions of the small intestine to facilitate further absorption. Thus, targeting the duodenum directly inhibits absorption from that region of the intestine and may also indirectly inhibit absorption from the ileum and/or jejunum.

As noted, the present devices are either introduced through a body cavity (e.g., the oral cavity) or are inserted directly into the small intestine through an abdominal incision. The devices can be configured so the energy emitting portion of the device is simply passed along a length of undisturbed intestinal lumen, in which case energy primarily reaches the peaks of the folds containing vascular and lymphatic structures, or passed along a length that has been expanded, in which case energy reaches an increased percentage of vascular and lymphatic structures during a procedure. In some cases, the small intestine will be treated through the entire length with a low percentage of vessels and ducts affected. In other cases, small sections of the small intestine may be treated using a larger applicator resulting in a greater percentage of vasculature and lymphatic treatment.

### BRIEF DESCRIPTION OF THE DRAWINGS

FIG. 1 illustrates a medical device for treating tissue within the small intestine. The device has a single tissue expander at the distal end with a smooth, conical exterior and four internal channels (for inflation of the tissue expander, energy delivery, visualization, and a working channel for electrolyte, coolant, or vacuum application).

FIG. 2 illustrates a medical device for treating tissue within the small intestine. The device has a single tissue expander at the distal end in the shape of an elongated balloon and four internal channels.

FIG. 3 illustrates a medical device for treating tissue within the small intestine. In this device, the energy-emitting element lies between a first tissue expander at the distal tip and a second tissue expander, which is also in the distal region (but more proximal to the operator). The tissue expanders are shaped differently, illustrating the heterogeneity of this element.

FIG. 4 illustrates a medical device for treating tissue within the small intestine. In this device, the energy-emitting element lies between a first tissue expander at the distal tip and a second tissue expander, which is also in the distal region of the device but more proximal to the operator. The tissue expanders in this embodiment are similarly shaped.

FIG. 5 illustrates a pair of medical devices for treating tissue within the small intestine. In the uppermost device (5A), the energy-emitting element is positioned between two differently shaped tissue expanders (the more proximal of the two being spherical). In the lower-most device (5B), both of the tissue expanders are spherical. Where the energy-emitting element is fixed and energy emission is unrestricted in direction, energy can emanate equally well in any or all directions from its point of origin (5C). Where the energy-emitting element is focused and moveable, energy emanates toward a focused point and can move or “sweep” around the intestinal lumen (5D).

FIG. 6 illustrates a medical device for treating tissue within the small intestine. The energy-emitting element is an active electrode band encircling or recessed within a spherical tissue expander. A device configured in this manner can be used for radiofrequency application of energy. The active electrode band serves as the active electrode for monopolar energy application. For bipolar energy, the spherical tissue expander can provide a return energy path. The spherical tissue expander can be transparent to facilitate viewing and illuminating the tissue.

FIG. 7 illustrates a control system for energy application to a subject with a device as described herein.

### DETAILED DESCRIPTION

The present invention provides compositions (i.e., devices and kits) and methods for applying energy to the small intestine (e.g., the internal, lumenal surface of the duodenum, ileum, and/or jejunum) to reduce the ability of that tissue to absorb calories from food. Although the invention is not limited by the physiological mechanisms underlying the reduction in caloric uptake, we expect that the present devices and methods impair blood vessels and lymphatic tissue in the treated areas, thereby reducing absorption of nutrients and reducing, in effect, the treated patient's caloric intake. Again, while the invention is not so limited, we hypothesize that the devices and treatments will not interrupt or impair the secretion of mucus, bile, or pancreatic enzymes, or their mixture with water to any significant or detrimental extent (allowing the bowel to function essentially normally). However, nutrients within a mixture of these fluids and digested food will be absorbed into the vascular system in a treated patient at a rate less than the normal rate of absorption (i.e., the rate in an untreated individual) and/or in an amount less than the normal amount absorbed.

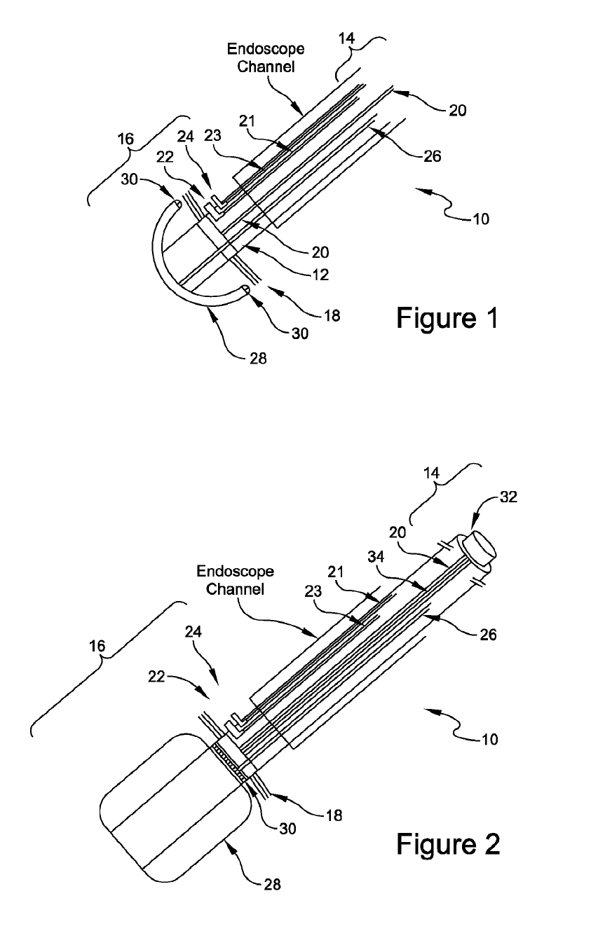

Referring to the figures, FIG. 1 illustrates a device 10 for applying energy to tissue within the intestinal lumen. The energy-application device 10 generally includes a housing 12, having a proximal end 14 and a distal end 16. Energy is shown emanating from the energy-emitting element 18 as energy from an energy source travels through the energy-delivery channel 20 from the proximal end 14 to the distal end 16 of the housing 12. The embodiment of the device 10 that is illustrated also includes a visualization channel 21, which terminates in a scope 22; a working channel 23, which terminates in a dispensing port 24; and an inflation channel 26, which terminates in a tissue expander 28. A sensor 30 is present at one or more points on the circumference of the tissue expander 28 to collect data, such as temperature, from the treatment environment. The energy-emitting element 18, the tissue expander 28, and any one or more of the visualization channel 21, scope 22, working channel 23, dispensing port 24, inflation channel 26, and tissue expander 28 may be fixed relative to one another and/or relative to the housing 12. Alternatively, the energy-emitting element 18, the tissue expander 28, and any one or more of the visualization channel 21, scope 22, working channel 23, dispensing port 24, inflation channel 26, and tissue expander 28 may be flexible, malleable, or flexibly or malleably interconnected so as to permit adjustment of their relative orientation or position relative to one another and/or relative to the housing 12. Preferably, the housing 12 is relatively rigid or semi-rigid (at least rigid enough to be advanced through an endoscope and the intestinal lumen).

A scope 22 is attached to or integrated with the visualization channel 21 at or near the distal end 16. Scope 22 collects imagery from within the intestinal lumen via an aperture, which imagery may then be output on a monitor or display. The imagery can provide visual confirmation of both the anatomical surroundings and the relative orientation of device 10. The physician can thus utilize the imagery as a visual aid in properly positioning the energy-emitting element 18.

In some embodiments, the scope 22 is a component of a fiber optic-based system that transmits images over an optical fiber within the visualization channel 21 to a display device (not shown). It should be understood, however, that other scopes, including ultrasound and infrared sensors, are useful and can be incorporated into device 10. The term “scope” is used herein to encompass all image capture devices, visualization devices, cameras, sensors, and any other element that captures and can transmit images, so long as the element is suitable (for example, in size and content) for surgical use.

While the position of the visualization channel 21 can vary, it may be preferable to position the visualization channel 21 such that the scope 22 is relatively unobstructed by any other element of the device 10 and such that the scope 22 can be rotated or otherwise adjusted to obtain images from a variety of field. For example, scope 22 can be rotated about the axis of the visualization channel 21 as a periscope is rotated.

Visualization of the intestinal lumen will be aided by a light source. Light may be provided by a light source on the present device or a light source on or inserted through an endoscope or similar surgical device through which the present device has been inserted.

One of ordinary skill in the art will understand that many if not all of the features and elements described in the context of FIG. 1 can be variously incorporated in other embodiments of the present device.

Referring to FIG. 2, device 10 may include a connector 32 at proximal end 14. Connector 32 may be configured to couple device 10 to a vacuum source (through which negative pressure may be applied to a subject's tissue and/or the intestinal lumen through working channel 24), a fluid delivery source and mechanism (through which an electrolyte solution or coolant may be applied to a subject's tissue through working channel 24), a control system, a power source, a data collection system, and the like, and any combination thereof. A handle may be attached that includes actuators or other control mechanisms for any of the systems, power sources, energy sources, or other sources to which device 10 is coupled via connector 26 (e.g., a switch to activate or deactivate energy, fluids, or coolant delivery to a subject's tissues). Devices including one or more handles and/or switches for regulating the delivery of energy or matter (e.g., an electrolyte solution) to device 10 are within the scope of the present invention.

In the embodiment of the device 10 that is illustrated in FIG. 2, energy is shown emanating from the energy-emitting element 18 as energy from an energy source travels through the energy-delivery channel 20 from the proximal end 14 to the distal end 16 of the housing 12. The energy-delivery channel 20 in this embodiment guides an energy conduit 34. The “conduit” is to be understood as broadly encompassing any material that facilitates the flow of energy from a source to the energy-emitting element 18 (e.g., an optical fiber). In this embodiment, the device 10 also includes a visualization channel 21, which terminates in a scope 22; a working channel 23, which terminates in a dispensing port 24; and an inflation channel 26, which terminates in a tissue expander 28. The sensor 30 is positioned between the energy-emitting element and the tissue expander 28. Information detected by the sensor 30 can be conveyed through the working channel 23 or otherwise transmitted to a control system or to the physician. Any of these elements can be fixed relative to one another and/or relative to the housing 12. Any one or more of these elements can be flexible, malleable, or flexibly or malleably interconnected. The tissue expander 28 at the distal end 16 is oblong or elongated in shape and may be rigid or expandable (e.g., inflatable).

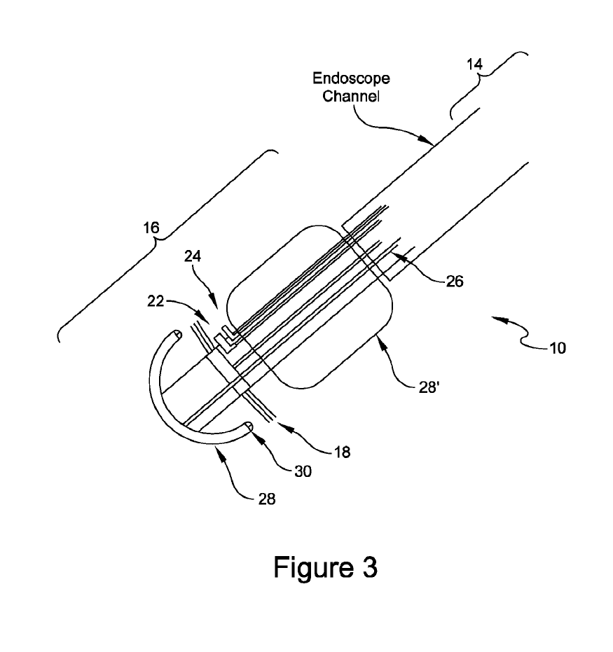

Referring to FIG. 3, an embodiment is illustrated in which the device 10 includes a first and a second tissue expander 28 and 28′ on either side of the energy-emitting element 18. The first and second tissue expanders 28 and 28′ assume different shapes. Embodiments in which the energy-emitting element lies between two tissue expanders may be preferable where more aggressive treatment is desired, as stretching the tissue on both sides of the energy-emitting element should expose a greater amount of the tissue to the emitted energy.

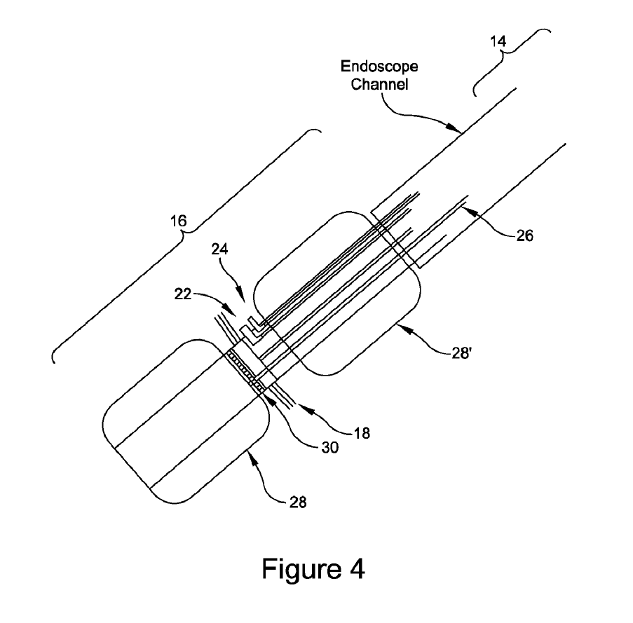

Referring to FIG. 4, an embodiment is illustrated in which the device 10 includes a first and a second tissue expander 28 and 28′ on either side of the energy-emitting element 18. The first and second tissue expanders 28 and 28′ are highly similar in size and shape. As noted, one may configure the energy-emitting element between two tissue expanders when more aggressive treatment is desired.

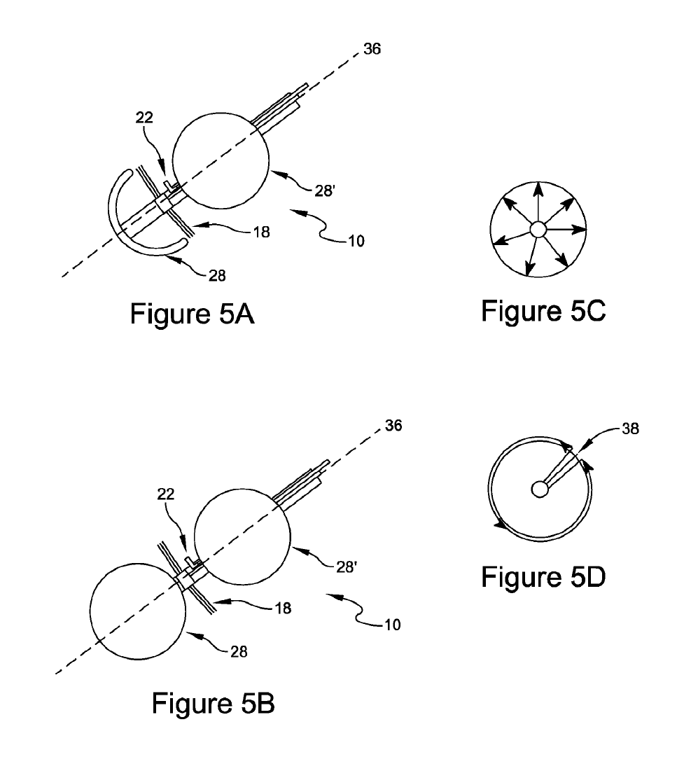

Referring to FIG. 5, an embodiment is illustrated in which the device 10 carries an energy-emitting element 18 positioned between two differently shaped tissue expanders 28 and 28′, the more proximal of which 28′ is spherical (5A) and extends well away from the central axis 36. In another embodiment, the tissue expanders 28 and 28′ are both spherical and may be inflatable. Where the energy-emitting element 18 is fixed and energy emission is unrestricted in direction (e.g., unshielded), energy can emanate equally well around the central axis 36 (as illustrated in FIG. 5C). Where the energy-emitting element 18 is focused, shielded, and/or moveable (e.g., rotatable around the central axis 36), energy emanates toward a focused point 38 and can move or “sweep” around the intestinal lumen (as illustrated in FIG. 5D). The energy (e.g., laser energy) can be projected directionally over a narrow arc (e.g., 30-60 degrees) or over all points of the arc (i.e., around) 360°. By way of analogy, the energy-emitting element illustrated by FIG. 5C is akin to a simple light bulb whereas the energy-emitting element illustrated by FIG. 5D is akin to a flashlight.

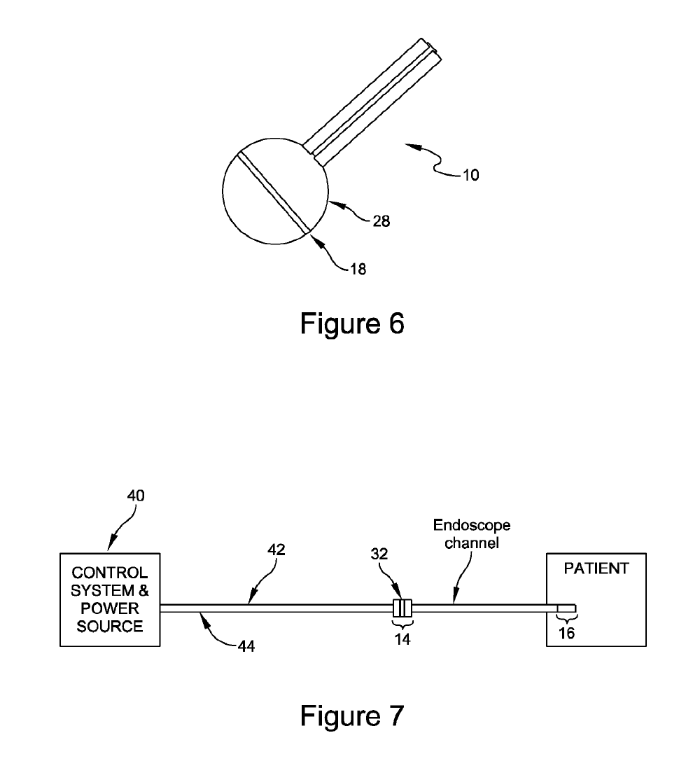

Referring to FIG. 6, an embodiment is illustrated in which the energy-emitting element 18 circumscribes or is integrated around the periphery of a tissue expander 28. The remaining elements of the device can be selected from those described herein.

Referring to FIG. 7, a system as described herein is illustrated which encompasses a control system and power source 40 operably linked via a conveyor 42 (e.g., wires, cables, or other conduits) to a proximal region 14 of the device and, optionally, to the connector 32. The device 10 optionally includes one or more handles or switches accessible to the physician in the region of, or extending from, the conveyor 42, connector 32, or proximal region 14 of the device 10, and these handles or switches (or the like) can be used to modulate the application of energy based on feedback received by the physician. Alternatively, the modulation can be automated by virtue of a connection between a feedback conveyor 44, conveying information from a sensor, as described herein, and the control system 40.

The positioning of the devices within a subject can be facilitated by insertion through an elongated, semi-ridged, flexible, and/or steerable fiber delivery system. Tissue expanders could be partially inflated prior to insertion into the small intestine as desired. Inflatable tissue expanders can be advantageous in that they can be inflated to various degrees, allowing the surgeon to customize the amount of the vascular system to be treated by adjusting the amount of expansion (greater inflation/expansion would expose a greater percentage of the intestinal wall for treatment). Once the energy-emitting element reaches the area to be treated, the tissue expander would then be expanded to the desired amount, and energy would be applied for a time sufficient to reduce the vascularization of the tissue to an extent that reduces nutrient uptake. The tissue expander can then be contracted (e.g., deflated) and moved to a new, untreated area where it would be re-expanded (e.g., re-inflated) prior to treatment of the new area.

Where the energy-emitting element is integral to a tissue expander, the material used for the tissue expander is preferably transparent to the emitted energy. For example, a laser with a wavelength of 577 nm could be used due to its high level of absorption into the blood vessels, and a material (e.g., a resin or polymer, such as plastic) can be used in the tissue expander that would not absorb the 577 nm laser light, allowing the energy to reach the target vasculature. This selectivity would also help prevent the laser from being absorbed into normal structural or connective tissue of the intestinal wall.

Where the tissue expander is at the far distal end (the tip) of the device, they may be referred to as an applicator tip, particularly where a circumferentially located ring electrode (for use with radiofrequency energy) is used as an energy-emitting element. The ring electrode could have an equatorial location in a spherical tissue expander to allow the electrode to rest directly against the intestinal wall at the point of treatment. This style tip can be used when the energy source is either a monopolar or bipolar radiofrequency energy source. If bipolar energy is used, the device can include a fine insulator between an equatorial electrode and each of the half sphere ends of the ball. Although a tip configured in this manner could be expandable, using a fixed size applicator may have advantages. For example, a fixed size applicator can be advanced through the intestine with the energy source on, and this could increase the speed of the treatment.

For the sake of clarity and labeling in the illustrations, the various channels within the present devices are not typically drawn to their full length (with the proximal ends generally staggered to aid identification). It will be clear from this description and one of ordinary skill in the art would understand that the channels generally traverse the entire longitudinal axis of the housing, operably connecting the proximal and distal ends of the devices.

The present invention can be used to treat subjects who are overweight or obese, including subjects who have tried and failed to lose weight by dieting and other behavioral modifications. A person who is overweight or obese is at risk for a number of health related issues, such as diabetes, atherosclerosis, coronary artery disease, myocardial infarction, hypertension, congestive heart failure, arthritis, sleep apnea, dyslipidemia, lipodystrophy, and cardiovascular accident. Thus, while we have characterized the devices and methods of the invention as devices and methods for promoting weight loss, they are useful in reducing the risk of many undesirable conditions associated with, or secondary to, excess body weight (including those listed above). Typically, a subject is considered overweight if his or her weight is at least or about 10% higher than a healthy norm (i.e., the top of a range considered to be a healthy norm), as defined by standardized height/weight charts, and considered obese if his or her weight is at least or about 30% or more above what is considered to be a healthy weight. Thus, subjects meeting these standards are candidates for treatment as described herein, unless there is a prevailing counterargument. One of ordinary skill in the art is able to determine whether or not a given subject is a good or poor candidate for treatment, and identifying a patient in need of treatment (e.g., by assessing height, weight, BMI, and other measurements) can be a step included in the present methods. While the methods of the invention can be applied to any mammal in need of treatment, the subjects will likely be human in the vast majority of cases. However, since the methods are minimally invasive and relatively inexpensive, veterinary application to animals such as domestic pets (e.g., cats and dogs) is also feasible. In recent years, the incidence of obesity has become more prevalent in people of all ages, including children and the elderly. The subjects amenable to treatment with the present methods may vary greatly in age and include children, teens, adults, and elderly men and women. Here again, the minimally invasive nature of the methods is an advantage. The present devices can readily be proportioned (e.g., in length and diameter) to accommodate any type of subject (e.g., a human child or adolescent, or a domesticated animal).

As is well known in the art, the small intestine is located in the abdominal cavity below the diaphragm and is positioned in the GI tract between the stomach and the large intestine. The small intestine is used for digestion of food and for mixing food with gastric juices to facilitate its breakdown. The stomach releases food into the duodenum (chyme), the first segment of the small intestine. Food enters the duodenum through the pyloric sphincter in amounts that the small intestine can digest. When full, the duodenum signals the stomach to stop emptying or transferring food. The duodenum receives pancreatic enzymes from the pancreas and bile from the liver and gallbladder. These fluids, which enter the duodenum through an opening called the sphincter of Oddi, are important in aiding digestion and absorption. When energy is applied to the duodenum, as described herein, this sphincter can be avoided. Peristalsis also aids digestion and absorption by churning up food and mixing it with intestinal secretions.

While the first few inches of the duodenal lining are smooth, the remainder of the lining has folds, small projections (villi), and even smaller projections (microvilli). These villi and microvilli increase the surface area of the duodenal lining, allowing for greater absorption of nutrients. The remainder of the small intestine, located below the duodenum, consists of the jejunum followed by the ileum. Turning movements facilitate absorption. Absorption is also enhanced by the vast surface area made up of folds, villi, and microvilli. As noted, the present methods can be applied to any area of the small intestine, although the duodenum may be favored.

The wall of the small intestine is anatomically divided into four layers. The mucosa is a membrane that lines the inside of the digestive tract. Materials in broken down food cross the mucosa to reach the bloodstream and are carried off to other parts of the body for storage or for chemical change. Although this process varies with different types of nutrients, all nutrients ultimately enter the body through vascular structures. Therefore, energy emitted by the present devices and delivered by the present methods can be delivered to any layer of the intestine that contains vascular structures that absorb nutrients and calories. For example, the energy-emitting element can be positioned on or near the surface of the inner layer and it may be configured such that emitted energy penetrates the mucosa and is delivered to the underlying vascular structures. For example, in the case of a laser delivery system, the wavelength of emitted light may be set such that it penetrates the mucosa and is absorbed by the vascular and/or lymphoid structures (e.g., laser energy in the 480 nm to 650 nm range is absorbed by the target chromophore while passing through the mucosa). The emitted energy may target blood within the vessels, the walls of the vascular structure, lymphatic ducts, or a combination of these tissue types.

FIGS. 1-5 illustrate devices in which a tissue expander lies distal to the energy-emitting element of the device, and FIG. 6 illustrates a device in which the tissue expander incorporates the energy-emitting element. In other embodiments, the tissue expander may be located only proximal to the energy-emitting element. In any configuration, the energy-emitting portion of the device may also include an element for additional diffusion of the emitted energy (an element that enhances a diffuse pattern) or a shield that preferentially directs the emitted energy to a more focused point on the tissue. In any embodiment, the energy-emitting element may be preceded and/or followed by a shaped tissue expander, which can be a spherical, elliptical, or elongated structure of either a fixed size and shape or an adjustable size and shape (e.g., inflatable). For example, the tissue expander(s) can be sized or adjusted in size to have a dimension (e.g., a cross-sectional diameter) about the size of the intestinal lumen or slightly larger to achieve the tissue expansion described herein; a tissue expander can distend the lumen so that more tissue is exposed to the emitted energy and/or the tissue is more evenly treated. When present, a shield around a portion of the energy-emitting element directs the emitted energy and can be used to achieve either a random or a specific treatment pattern. For example, the focusing element can rotate the energy source such that a beam of energy is rotated 360° around a central axis. If heat is used to treat the tissue, a heat source (e.g., a fiber-optic) may terminate in a metallic device that will distribute the heat energy that it absorbs. Spatially, the distribution can be uneven or substantially even. The metallic portion of the device or other heat diffusor can be designed as a cylindrical or spherical device that is moved or rolled against the intestinal wall. It may also be fashioned as a semi-circular or cap-type structure. The metallic element may be made of aluminum, stainless steel, silver, gold or any other electrically conductive material. The material may be an alloy (e.g., stainless steel), and one or more of the materials, including those just listed can be mixed or used in combination to form the applicator portion of a device. Alternatively, nonconductive material such as carbon fiber, fiberglass, or plastic may be used. If metallization is performed on the carbon fiber, fiberglass, or plastic, it may be continuous or may alternate with aluminum or other conductive mesh or wires. Further, the energy-emitting portion of the device may be rigid, semi-rigid or more substantially flexible. Moreover, the element may be solid or hollow. For example, electrodes may be passed through a hollow wire to a metalized tip for application to the tissue.

Once the desired amount of effect is obtained, the energy may be interrupted (i.e., terminated for a time). Depending on the exact configuration of the energy-applicator device, all or a portion of the device may be removed from its location near the treated tissue and redeployed to another area of the small intestine. For example, either the device as a whole or a potion thereof (e.g., the energy-emitting element) may be withdrawn, withdrawn and then moved to a new area, or simply advanced to a new area. During this process, visualization can be maintained through a viewing port and visualization channel contained either within the device or within a laparoscope, flexible catheter, or endoscope, through which the device has been inserted (e.g., through the esophagus, stomach, and into the small intestine).

As noted, the device can be deployed through either an open surgical procedure, through one or more minimal incisions, or through a body orifice, such as the mouth. In the case of an oral entry, a flexible scope carrying the device, optionally with its visualization, illumination, and temperature-adjustment channel(s) and sensor elements, can enter through the esophagus, passing through the stomach and into the small intestine. The flexible (i.e., non-rigid) scope could then be gradually moved through the small intestine treating the desired area(s), with the present device deployed from therein. The device can also be brought into contact with the small intestine through the rectum. Alternatively, it may be desirable to use a trocar to enter the abdominal cavity. A flexible scope would then be advanced to the beginning or some more distal portion of the small intestine where an incision would be made allowing the scope to be advanced directly into the small intestine. In one treatment procedure, a trocar is used to create an incision point in the abdominal wall. An instrument such as a laparoscope is then inserted and advanced to a region of the small intestine (e.g., the proximal section). Using standard surgical instruments an incision is then made into the small intestine providing direct access to the lumen of the small intestine. Once access into the small intestine is gained, the applicator can then be advanced into the small intestine and positioned in any part of the small intestine for treatment using one of the methods described above.

Energy may be applied together with an electrolyte solution, which can be delivered, for example, by way of a channel running through the long axis of the device (e.g., a working channel). The solution can also be contained in a reservoir within the device and delivered to the applicator region of the device in order to facilitate energy transfer from the energy-emitting element to the tissue. The electrolyte solution can be preheated to a selected temperature and modified as necessary.

Generally, the energy conduit can be a wire, waveguide, fiberoptic (or optical fiber), and the energy delivered can be interstitial, monopolar, bipolar, or dipolar. To reduce the risk of possible overheating and to carry away unwanted heat, a cooling aluminum can be positioned in the device. For example, the electrode or light guide can include a cooling lumen that is contiguous with the source of cooling fluid or gas. If a gas or air-cooled gap region is used, the tissue may be cooled, for example, by air-conditioned or room air or other gas directed to the tissue. The cooling liquid or gas may be applied continuously or intermittently as required to maintain the temperature of the tissue.

During treatment, the methods can be conducted under a feedback control, which can be accomplished by visualization, impedance, and ultrasound, with temperature measurement. One of ordinary skill in the art would understand the common instruments used for these feedback controls. If temperature measurement is used, the device can either be external to the energy delivery device or included within the energy delivery device. Temperature measurement can be accomplished by the use of one or more thermal sensors, such as infrared, thermistor, semiconductor temperature sensor, non-contact infrared detectors, or fiber-optic temperature sensors.

Similarly, one or more impedance sensors could be used for feedback control if radio frequency is used. Current and voltage are used to calculate impedance. The power, phase, amplitude, wavelength, frequency, pulse configuration, and pulse width may be computer controlled using feedback signals. If a laser is used, the power applied, amplitude, pulse configuration, and duty cycle may be regulated under feedback control through a temperature sensor.

The methods of the present invention can provide a minor reduction of nutrient absorption or a significant reduction of nutrient absorption depending on the extent to which the small intestine is treated. In certain circumstances, it may be desirable to reduce the amount of nutrients and calories that are absorbed to only a certain point and then reduce them further in subsequent treatments if need be.

As noted, the extent of vasculature treated depends generally on several factors, including the amount of the tissue treated and the power, frequency, amplitude, pulse configuration, and pulse width of the energy applied. If laser is used the power can be in the range of about 1 to 100 Watts/cm² (at least or about 25, 30, 35, or 40 watts/cm²). The wavelength can affect the method of the present invention by varying the efficiency of the absorption into the target chromophore, which, in the present methods, is blood contained in the vascular structure of the intestinal wall and the vascular structure itself. The pulse configuration can affect the method of the present invention by applying the energy gradually or instantaneously with either an abrupt or gradual reduction of energy. The pulse configuration can be in the range of about 1 pps to about 1000 pps (e.g., at least or about 5, 10, 12, 15, or 20 pps). The pulse width can be in the range of about 1 microsecond to about 1 sec (e.g., at least or about 0.1, 0.5, 1.0, 1.5, or 2.0 sec). Intermediate ranges of the figures just described are also useful within the methods of the present invention. For example, power in the range of 10-25 watts/cm² can be used, as can a pulse width of 0.01-100 ms.

The applicators used for the procedures described herein can vary in design depending on the type of energy used and the percentage of target tissue expected to be treated. A simple laser fiber, housed with a lens on or near the distal tip of the applicator, can be inserted into the lumen of the small intestine with no direct visualization (e.g., under fluoroscopic image control). The fiber would be advanced through the intestine or a portion thereof (e.g., a length of about 2-6 inches) while treatment is accomplished. The laser fiber and lens can be a part of (e.g., affixed within) a centering ring or ball, which provides greater control of the position of the fiber during treatment. With these applicators, little or no expansion of the intestinal wall would be created, thus allowing for treatment of patients in which a low percentage of tissue (e.g., about 5-35% of the intestine) is to be treated.

It may also be advantageous to maintain a substantially constant and consistent amount of pressure across an area to be treated with an applicator. To maintain consistent application of energy in the treatment area, a gas or an inflatable object may be placed in the abdominal cavity to apply counter pressure to the applicator.

As noted, the present devices include one or more tissue expanders, which can be variously configured. For example, the expander can be shaped symmetrically, as a ring, or asymmetrically, for example as an irregularly-shaped cavity, either of which can be expanded to generate a structure that exerts pressure against the intestinal lumen (e.g., through mechanical expansion or by inflation; a “balloon”). The expander can be used to expose more of the tissue of the intestinal wall to the energy-emitting element. A device with an expanded balloon (e.g., expanded to about the diameter of the intestinal lumen or slightly more) provides not only additional access to the blood vessels vascularizing the intestinal tissue but also “unfolds” the normal folds of the intestinal wall, thereby exposing a larger area of vessels to be treated and increasing the total amount of vasculature treated. Because of the shape of the small intestine and the pliability of the wall of the small intestine, this object may also provide access to areas of the small intestine that may not otherwise be contacted by the energy.

The tissue expander in the present devices can be made from any of the materials used for the balloon-portion of devices for other minimally invasive procedures. These materials can withstand high pressure and yet have thin walls, high strength, and a small profile. The tissue expander can assume a wide range of diameters, lengths, and shapes, and can be custom formed if necessary for optimum expansion of a portion of the intestinal lumen. The material can be a high-pressure, non-elastic material or a lower pressure elastomeric balloon made, for example, of latex or silicone. For example, a balloon tissue expander can be formed from polyvinyl chloride, crosslinked polyethylene or another polyolefin, nylon, polyurethane, or PET (polyethylene terephthalate).

Alternatively, a vacuum may be used to draw the intestinal wall nearer to the energy-emitting element, thereby facilitating access to portions of the intestine that are harder to reach. The vacuum may be applied through a working channel of the device, as illustrated in the accompanying drawings. In such an instance, the device may contain a gasket that allows the vacuum to be in contact with the tissue and to hold the intestinal wall in place as an energy-emitting element (e.g., the terminus of one or more optical fibers) is placed near or against the intestinal wall to apply the energy. A vacuum can also be applied independently of the present device. For example, an endoscope may include a port through which vacuum or negative pressure can be applied.

The present methods can be combined with other therapies, such as dietary counseling, hypnosis, behavior modification, and pharmacological intervention.

The components of the devices described herein can be attached or assembled through standard electromechanical couplings known in the art or readily understandable by one of ordinary skill in the art.

A number of embodiments of the invention have been described. Nevertheless, it will be understood that various modifications may be made without departing from the spirit and scope of the invention. Accordingly, other embodiments are within the scope of the following claims.

1. A medical device for delivering energy to the lumen of the intestine, the device comprising:
(a) an elongated housing having a proximal end and a distal end;
(b) an energy-delivery channel, wherein the energy-delivery channel extends from the proximal end of the housing toward the distal end; is configured to transport energy or guide an energy conduit; and terminates beyond the distal end of the housing in an energy-emitting element; and
(c) a tissue expander at the distal end of the housing.

2. The medical device of claim 1, wherein the elongated housing is rigid or semi-rigid; is about 1 foot to about 25 feet long; and has a diameter suitable for insertion through a working channel of an endoscope or a trocar.

3. The medical device of claim 1, wherein the energy conduit comprises an optical fiber, wire, or transducer.

4. The medical device of claim 1, wherein the energy delivery channel or the energy conduit comprises a connector for attaching the channel or the conduit to an energy source.

5. The medical device of claim 4, wherein the energy source is a laser, radiofrequency generator, ultrasound generator, or cryogenic probe.

6. The medical device of claim 1, wherein the energy-emitting element is fixed such that energy emanates from the element in a diffuse pattern.

7. The medical device of claim 1, wherein the energy-emitting element is focused and moveable such that energy emanates from the element toward a focused point, which point can vary as the element is moved.

8. The medical device of claim 1, further comprising a second tissue expander, wherein the first tissue expander is located distal to the energy-emitting element and the second tissue expander is located proximal to the energy-emitting element.

9. The medical device of claim 1 or claim 8, wherein the first tissue expander and/or the second tissue expander is shaped as a sphere, an ellipse, a ring, or a cone.

10. The medical device of claim 1 or claim 8, wherein the first tissue expander and/or the second tissue expander is an inflatable balloon.

11. The medical device of claim 1, further comprising a visualization channel, wherein the visualization channel runs substantially parallel to the energy-delivery channel and comprises a scope, which is optionally moveable, attached to or integrated with the visualization channel at or near the distal end of the visualization channel.

12. The medical device of claim 11, wherein an optical element is positioned over the aperture.

13. The medical device of claim 12, wherein the optical element is a lens or filter.

14. The medical device of claim 1, further comprising a temperature-adjustment channel, wherein the temperature-adjustment channel runs substantially parallel to the energy-delivery channel and is configured to transport a fluid or gas of a given temperature to the tissue to which energy has been applied by the device.

15. The medical device of claim 1, further comprising at or near the distal end of the housing, a sensor for determining temperature.

16. A method for reducing the amount of nutrients that are absorbed into the vascular system of the small intestine of a subject, the method comprising:
providing the medical device of claim 1;
positioning the energy-emitting element of the device within the lumen of the small intestine of the subject; and
applying energy from the device to the internal surface of the small intestine, wherein the energy is of a type and delivered for a time sufficient to inhibit the absorption of nutrients from the treated portion of the small intestine.

17. The method of claim 16, wherein positioning the energy delivery device comprises inserting the energy-emitting element of the device into the intestine through a laproscope positioned in the subject's abdominal cavity.

18. The method of claim 16, wherein the medical device delivers laser energy and the method further comprises application of an electrolyte solution between the energy-emitting element and the surface of the intestinal tissue being treated.

19. The method of claim 16, wherein the medical device delivers laser energy and comprises or is connected to a power source supplying energy with a power of about 0.1 to about 50 watts/cm².

20. The method of claim 19, wherein the power is at least or about 25, 30, 35 or 40 watts/cm².

21.-37. (canceled)
